(19) United States
(12) Patent Application Publication
(10) Pub. No.: US 2021/0005299 A1
(43) Pub. Date: Jan. 7, 2021

(54) SYSTEM AND METHOD FOR IMPROVING TREATMENT OF A CHRONIC DISEASE OF A PATIENT

(71) Applicant: Patientory, Inc., Atlanta, GA (US)

(72) Inventor: Chrissa Tanelia McFarlane, Atlanta, GA (US)

(21) Appl. No.: 16/583,147

(22) Filed: Sep. 25, 2019

Related U.S. Application Data
(60) Provisional application No. 62/736,387, filed on Sep. 25, 2018.

(51) Int. Cl. G16H 20/00; G16H 80/00; G16H 10/60; H04L 9/06; H04L 9/32

(52) U.S. Cl. CPC G16H 20/00; G16H 80/00; H04L 2209/38; H04L 9/0637; H04L 9/3263; G16H 10/60

(57) ABSTRACT

An HIE system and a method for improving treatment of a chronic disease of a patient are described. The method comprises providing a user device for allowing a patient to connect to a health care network implemented over blockchain and to manage access of patient data stored over the blockchain. The method further comprises providing a physician network device for allowing a physician to connect to the health care network. A request may be received from the physician for accessing the patient data and a first parameter related to the patient. The first parameter may be correlated with patients' data to identify correlated data points relevant for the patient. The correlated data points may be sent to the physician, for being used towards the treatment of the chronic disease.

### OTHER RELATED APPLICATIONS

The present application is a U.S. Non-Provisional patent application claiming priority of U.S. Provisional Patent Application Ser. No. 62/736,387 filed on Sep. 25, 2018, which is hereby incorporated by reference.

### BACKGROUND OF THE INVENTION

### 1. Field of the Invention

The present disclosure is generally related to data processing in a healthcare network implemented over blockchain, and more particularly related to data processing for identifying treatment of a chronic disease of a patient.

### 2. Description of the Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption define storage of all information in a block wise manner. The blocks are added to the blockchain in a linear and chronological order. As the entirety of sensitive information present on the blockchain (data) is present over several blocks, it is difficult to gather the data at a single place and thereupon analyze or process such data for meeting specific requirements while also managing data access by interested users. Therefore, there exists a need for more effectively and efficiently managing access of the data and processing the data to facilitate user's requirements.

Applicant believes that a related reference corresponds to U.S. Pat. No. 8,170,887B2 issued for a system and method for providing continuous, expert network care services from a remote location(s) to geographically dispersed healthcare locations. However, the reference differs from the present invention because it fails to address the issue of providing a health interchange exchange system utilized to improve the treatment of a chronic disease found in a patient. Additionally, the system utilizes cloud networks to efficiently store sensitive information in an encrypted manner. The present invention addresses these issues by providing a system and method that includes a health interchange exchange system that is utilized for improving the treatment of a chronic disease in a patient.

Other documents describing the closest subject matter provide for a number of more or less complicated features that fail to solve the problem in an efficient and economical way. None of these patents suggest the novel features of the present invention.

### SUMMARY OF THE INVENTION

It is one of the objects of the present invention to provide a system and method for improving treatment of a chronic disease of a patient providing a user device for allowing a patient to connect to a health care network implemented over blockchain and to manage access of patient data stored over the blockchain.

It is another object of this invention to provide a system and method for improving treatment of a chronic disease of a patient providing a physician network device for allowing a physician to connect to the health care network.

It is still another object of the present invention to provide a system and method for improving treatment of a chronic disease of a patient that allows for a request to be received from the physician for accessing the patient data and a first parameter related to the patient.

It is yet another object of this invention to provide such a device that is inexpensive to implement and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

The above and other related objects in view, the invention consists in the details of construction and combination of parts as will be more fully understood from the following description, when read in conjunction with the accompanying drawings in which:

FIG. 1 illustrates a network connection diagram 100 of a Health Information Exchange (HIE) system for improving treatment of a chronic disease of a patient, according to various embodiments.

FIG. 2 illustrates a method for symmetric encryption of data, according to various embodiments.

FIG. 2A illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in the health care network implemented specifically over a blockchain network, according to various embodiments.

FIG. 6 illustrates exemplary data stored in a treatment database, according to various embodiments.

FIG. 6A illustrates exemplary data stored in a patient database, according to various embodiments.

FIG. 6B illustrates exemplary data stored in a recommendation database, according to various embodiments.

FIG. 7 illustrates a flowchart showing a method performed by a patient module, according to various embodiments.

FIG. 8 illustrates a flowchart showing a method performed by a treatment network base module, according to various embodiments.

FIG. 9 illustrates a flowchart showing a method performed by a data collection module, according to various embodiments.

FIG. 10 illustrates a flowchart showing a method performed by a workflow machine learning module, according to various embodiments.

FIG. 11 illustrates exemplary data stored in the workflow machine learning module, according to various embodiments.

FIG. 11A additionally illustrates exemplary data stored in the workflow machine learning module, according to various embodiments.

FIG. 12 illustrates a flowchart showing a method performed by a physician network base module, according to various embodiments.

### DETAILED DESCRIPTION OF THE EMBODIMENTS OF THE INVENTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of various embodiments of the present disclosure, various embodiments of the systems and methods will be described.

Embodiments of the present disclosure will be described more fully hereinafter with reference to the accompanying drawings in which like numerals may represent like elements throughout the several figures, and in which various example embodiments are shown. Various embodiments may, however, be embodied in many different forms and should not be construed as limited to the embodiments set forth herein. The examples set forth herein are non-limiting examples and are merely examples among other possible examples.

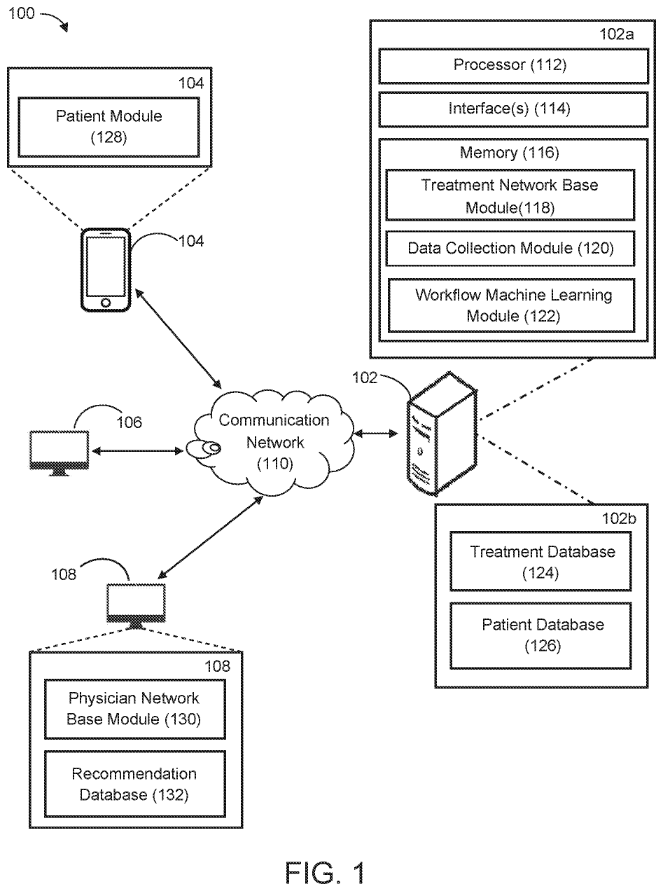

FIG. 1 illustrates a network connection diagram 100 of a Health Information Exchange (HIE) system 102 for improving treatment of a chronic disease of a patient. The HIE system 102 may comprise one or more user interfaces. The one or more user interfaces may be accessed by one or more users via one or more devices. The one or more device may comprise, for example, a user device 104, a doctor's device 106, and a physician network device 108. The HIE system 102 may be connected with the user device 104, the doctor's device 106, and the physician network device 108, through a communication network 110.

The communication network 110 may be a wired and/or a wireless network. The communication network 110, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may comprise a group of components 102a for improving the treatment of chronic diseases. The group of components 102a may include a processor 112, interface(s) 114, and a memory 116. The memory 116 may include a treatment network base module 118, a data collection module 120, and a workflow machine learning module 122. Further, the HIE system 102 may include or may be connected with a group of databases 102b which may include a treatment database 124 and a patient database 126.

The processor 112 may execute an algorithm stored in the memory 116 for improving the treatment of the chronic diseases. The processor 112 may also be configured to decode and execute any instructions received from one or more other electronic devices or server(s). The processor 112 may include one or more general purpose processors (e.g., microprocessors) and/or one or more special purpose processors (e.g., digital signal processors (DSPs) or System On Chips (SOCs), Field Programmable Gate Arrays (FPGAs), or Application-Specific Integrated Circuits (ASICs)). The processor 112 may be configured to execute one or more computer-readable program instructions, such as program instructions to carry out any of the functions described in this description.

The interface(s) 114 may help an operator to interact with the HIE system 102. The interface(s) 114 may either accept inputs from users or provide outputs to the users, or may perform both the actions. In various embodiments, a user can interact with the interface(s) 114 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or any combination of the above. Further, the interface(s) 114 may be implemented as a Command Line Interface (CLI), a Graphical User Interface (GUI), a voice interface, or a web-based user-interface.

The memory 116 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. The memory 116 may comprise modules implemented as a program. As mentioned above, the memory 116 may comprise the treatment network base module 118, the data collection module 120, and the workflow machine learning module 122.

In various embodiments, several users may interact with the HIE system 102, using the user device 104. The user device 104 may include a patient module 128. Although a single user device has been illustrated, several user devices could similarly be connected to the communication network 110. Further, each of the user devices may have a device ID. In various embodiments, the device ID may be a unique identification code such as an International Mobile Equipment Identity (IMEI) code or a product serial number. It should be noted that a user may use a single user device or multiple user devices. Further, multiple users may use a single user device or multiple user devices. Further, the one or more users may receive and/or provide healthcare related products and services. The one or more users may include, for example and not limited to, patients, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but not limited to, a computer, a laptop, a tablet, a mobile phone, a smartphone, or a smart watch. In various embodiments, the user device 104 may include an imaging device that may be configured to capture a visual graphical element. The visual graphical element may be, for example but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In another embodiment, the imaging device may be located external to the user device 104. In various embodiments, the imaging device may be connected to the user device 104 wirelessly or via a cable. It should be noted that image data of the visual graphical element may be transmitted to the user device 104 via the communication network 110.

In various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, the camera may be controlled by a processor natively embedded in the user device 104. In various embodiments, the imaging device may include a screen capturing software (for example, screenshot) that may be configured to capture and/or scan the QR code on a screen of the user device 104.

In various embodiments, the HIE system 102 may communicate with the doctor's device 106, through the communication network 110. The doctor's device 106 may be accessed by doctors for receiving data related to the patients for improving the treatment of the chronic diseases. In various embodiments, the HIE system 102 may communicate with the physician network device 108, through the communication network 110. The physician network device 108 may be operated by the physicians. A physician may be an individual belonging to one of hospitals, insurance companies, Contract Research Organizations (CROs), and drug companies. Further, the physician network device 108 may include a physician network base module 130 and a recommendation database 132. In various embodiments, the physician network device 108 may include a user interface i.e., physician network Graphical User Interface (GUI) to allowing the physicians to interact with the physician network device 108.

In various embodiments, the group of databases 102b may be implemented over a blockchain network (such as a PTOYNet blockchain network or a PTOYNet Ethereum™ Blockchain network) and may be present as different databases installed at different locations. The group of databases 102b, which may include the treatment database 124 and the patient database 126, may be configured to store data belonging to different users and data required for functioning of the HIE system 102. Different databases may be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. In various embodiments, the data may represent the results of one medical test in a series of multiple medical tests.

In various embodiments, the group of databases 102b may operate collectively or individually. Further, the group of databases 102b may store data as tables, objects, or other data structures. Further, the group of databases 102b may be configured to store data retrieved or processed by the HIE system 102. The data may include, but not limited to, a patient medical history, medical charts, medications, prescriptions, immunizations, test results, allergies, insurance provider, or billing information. Further, the data may be time-dependent and piece-wise. Further, the data may represent a subset of data for each patient. In various embodiments, the data may represent results of a medical test in a series of multiple medical tests. Further, the data may be securely stored. In various embodiments, the data may be encrypted.

In various embodiments, the workflow machine learning module 122 of the HIE system 102 may be used to analyze care treatment plans for the chronic diseases. Based on the analysis of the care treatment plans, diet, medications, disease, or medical history of a patient, a recommend care plan may be provided to a physician. Further, the workflow machine learning module 122 may be used where timing of events is analyzed in order to assess nature of an event or lack thereof of events. In various embodiments, the workflow machine learning module 122 may aggregate data on a time duration that a radiologist spends to review X-rays for different diseases, and thereby compute an average time duration for each type of X-ray. Such method may enable the workflow machine learning module 122 with respect to the patient and hospital relationship and allow the workflow machine learning module 122 to determine average time duration needed to review certain types of X-rays. It should be noted that the data may deduce that if a physician does not look at broken leg of a patient within two hours, then something is wrong, and thereby alerting the physician.

In various embodiments, an analytical system and method used to determine the location at which the next workflow is created, based on the demographics, workflow, and specialty of the hospital. Once a workflow for a cardiologist is obtained, then the workflow may be replicated for use at another hospital. Such generic workflow improvement may be stored on an external server, outside of the blockchain. Further, a hospital may have, for example one server, but the hospital may have many different blocks for that one server since the hospital may want to store specialty-specific data on different servers. In various embodiments, cardiology data may be stored on one server and oncology data may be stored on another server.

In various embodiments, the HIE system 102 provides a proprietary feedback loop from the blockchain to the user-entity, i.e., hospital. In various embodiments, when the physician wishes to review a patient's X-Ray, the patient provides the physician with a public key. Further, the physician uses the public key to access the X-Ray through the interface of the physician, which opens up the blockchain's private key to grab the X-Ray and sends the X-Ray back to the physician. Thereafter, when the physician accesses the X-Ray, the data goes back to the blockchain platform into a hospital's proprietary workflow so that the hospital is aware that the physician looked at the X-ray.

In various embodiments, information stored in the group of databases 102b may be accessed based on users' identities and/or the users' authorities. The users' identities may be verified in one or more ways such as, but not limited to, biometric authentication (or bioauthentication), password or PIN information, user device registrations, a second-level authentication, or a third-level authentication. In various embodiments, the users' identities may be verified by the HIE system 102. Information provided by the users in a real-time may be used, by the HIE system 102, to confirm the users' identities. In various embodiments, the users' identities may be verified using a name, a password, one or security questions, or any combination thereof. In various embodiments, a user may be identified using an encryption key and/or a decryption key.

In various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level subsystem may be encrypted. In various embodiments, the second level subsystem may be implemented over a blockchain network (such as a PTOYNet blockchain network). In various embodiments, the PTOYNet blockchain network may be used to implement smart contracts.

In an exemplary scenario, a primary care physician may input data into the HIE system 102 using the user device 104. The data may be processed by the first level subsystem and the second level subsystem. This may be done successively. The data may be stored on the first level subsystem and/or the second level subsystem of the HIE system 102. This may be done successively. The data may include, but not limited to, one or more instructions to a patient to see a physician specialist. Further, the data may be stored in one or more blockchains of the second level subsystem. The patient may be able to access the data relating to the patient's care provided by the primary care physician. This may be done successively. The patient may be able to retrieve the data using the user device 104 of the patient. This may be done successively.

In accordance with various embodiments, the patient may communicate with the physician specialist using the HIE system 102. It should be noted that the physician specialist may be able to access the data of the patient from the first level subsystem and/or the second level subsystem. Further, the physician specialist may be able to communicate with the patient. It should be noted that some, all (or substantially all) communications between the primary care physician, the physician specialist and the patient may be stored and may be accessible on a blockchain network.

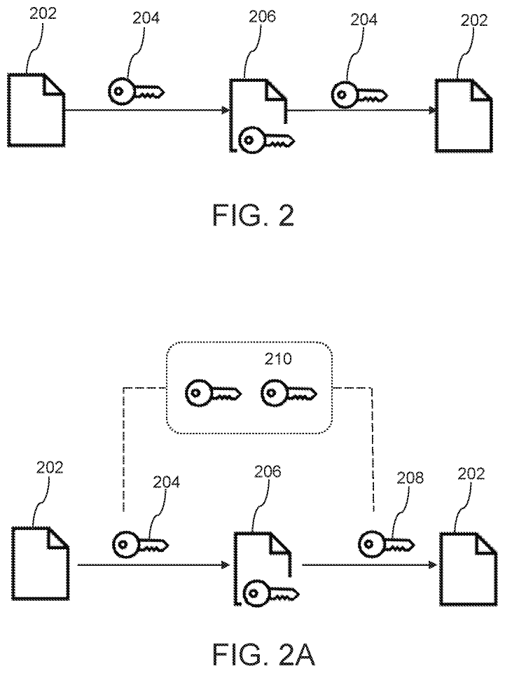

FIG. 2 illustrates a method for symmetric encryption of data, according to various embodiments. Original data 202 may be encrypted using a key 204 to obtain an encrypted data 206. Encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2A illustrates a method for asymmetric encryption of data, according to various embodiments. Original data 202 may be encrypted using a key 204 to obtain encrypted data 206. Encrypted data 206 may be decrypted using another key 208 to obtain the original data 202. It should be noted that encryption and decryption of the data may be performed using different keys e.g., a key pair 210.

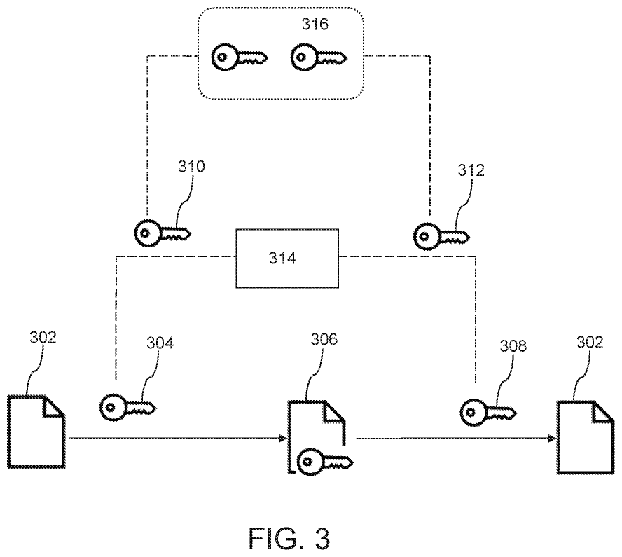

FIG. 3 illustrates a method for hybrid encryption of data, according to various embodiments. Both symmetric encryption and asymmetric encryption techniques may be used in tandem. In various embodiments, the symmetric encryption technique may be used to encrypt data 302 using a symmetric key 304 for producing encrypted data 306. The encrypted data 306 may be decrypted using another symmetric key 308 for obtaining data 302. Further, a public key 310 may be used to encrypt the symmetric key 304 and a private key 312 may be used to encrypt the symmetric key 308, stored as an encrypted key 314. The public key 310 and the private key 312 may for a key pair 316.

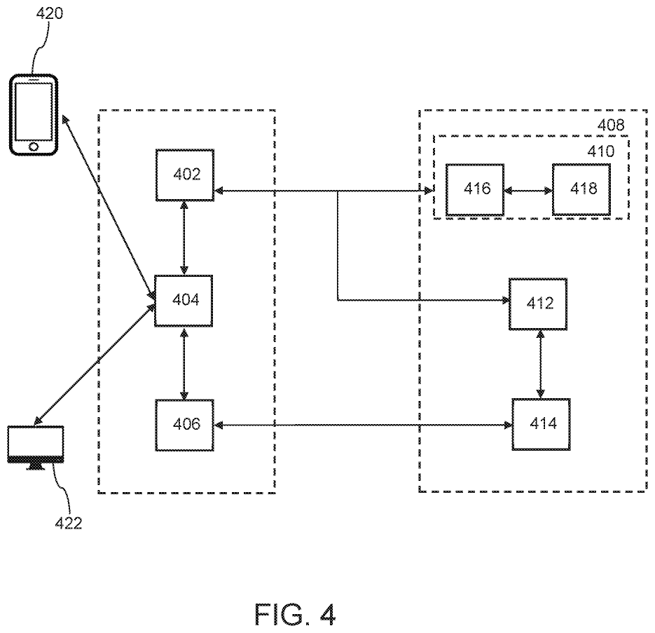

In accordance with various embodiments, referring to FIG. 4 illustrating an example of a system for storing and accessing data in a health care network, the first level subsystem may include a core service component 402 and a Remote Procedure Call (RPC) component 404. On the other hand, the second level subsystem may include a blockchain node component 406 (e.g., quorum blockchain node component 406). In an alternate embodiment, the first level subsystem may include the core service component 402, and the second level subsystem may include the RPC component 404 and the quorum blockchain node component 406. Further, the core service component 402 of the first level subsystem may be present in communication with third-party servers and databases of a hospital computing network 408. The hospital computing network 408 may include an Interplanetary File System (IPFS) module 410, an EHR synchronization service 412, and a blockchain node 414 (e.g., quorum blockchain node 414). Further, the IPFS module 410 may include IPFS manager 416 and an IPFS node 418. The quorum blockchain node component 406 of the second level subsystem may communicate with the quorum blockchain node 414 of the hospital computing network 408. Patients may access the health care network for storing data through the user device 104, and a representative of a hospital may access the health care network through another user device.

In accordance with various embodiments, the representative of the hospital may want to synchronize Electronic Health Record (EHR) data of a patient. The first level subsystem and the second level subsystem may ask the patient for permission to allow a representative of the hospital to store the EHR data of the patient, through the IPFS module 410. This may be done successively. Based at least on the permission granted by the patient, a signed transaction may be created to confirm the permission of the hospital to store the EHR data. Further, the signed transaction may activate a smart contract that may add hospital identification information such as a blockchain address to a list of permitted users.

In accordance with various embodiments, the signed transaction may be transmitted from the user device to the RPC component 404 of the first level subsystem and/or the second level subsystem. The RPC component 404 may communicate the signed transaction to the quorum blockchain node component 406 of the second level subsystem. This may be done successively. The quorum blockchain node component 406 may activate one or more smart contracts. This may be done successively. Thereafter, the quorum blockchain node component 406 may revise a state of one or more blockchains.

In accordance with various embodiments, based at least on the permission granted by the patient, the EHR synchronization service may obtain a list of patients from the RPC component 404. The EHR synchronization service may confirm whether the patient has granted permission. Based at least on the permission, the first level subsystem and the second level subsystem may obtain the EHR data and may calculate a hash function for the EHR data. The HIE system 102 may match the hash function of the EHR data with a hash function for the patient blockchain on the quorum blockchain node component 406 of the second level subsystem. This may be done successively. If the hash function of the EHR data matches with the hash function for the patient blockchain on the quorum blockchain node component 406 of the second level subsystem, the EHR data of the patient may remain unchanged.

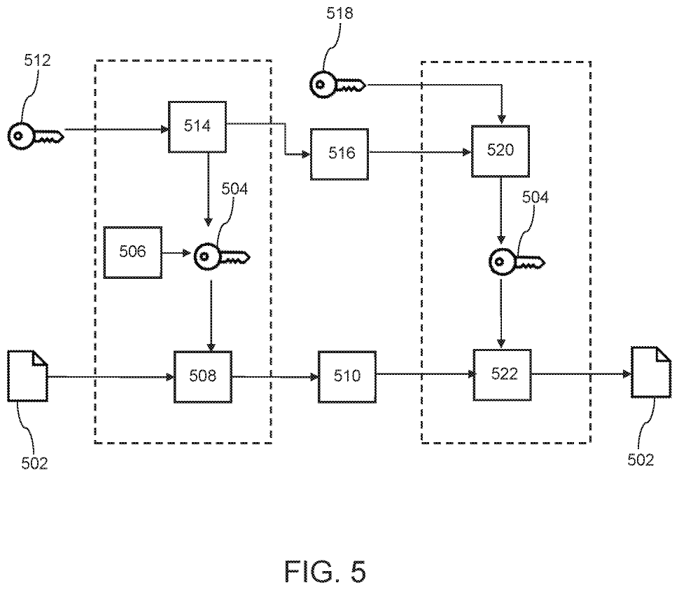

In accordance with various embodiments, referring to FIG. 5 illustrating a system for storing and accessing data in a health care network implemented specifically over a blockchain network (such as a PTOYNet blockchain network or a PTOYNet Ethereum™ Blockchain network), the HIE system 102 may execute an application for determining permission from the user for obtaining EHR data 502. In various embodiments, if the user grants the permission, the HIE system 102 may obtain the EHR data 502 for calculating a hash function for the EHR data 502. The HIE system 102 may match the hash function of the EHR data 502 with a hash function for the user blockchain on the quorum blockchain node of the second level sub-system. In various embodiments, if the two hash matches, there is no change to the user's EHR data 502. In various embodiments, if the two hash functions do not match, the HIE system 102 may generate a random string e.g., secret key 504, through a random key generator 506. The secret key 504 may be used for Advanced Encryption Standard (AES) encryption of the EHR data 502, in an AES encryptor 508, for generating encrypted EHR data 510.

In accordance with various embodiments, the secret key 504 may then be encrypted by, for example, a Rivest-Shamir-Adleman (RSA) public key 512 of the patient, in an RSA encryptor 514, to generate an encrypted secret key 516. The HIE system 102 may also send the encrypted EHR data 510 to the core service component 402 for forwarding the data to the IPFS manager 416 of the hospital computing network 408 for storage. The IPFS manager 416 may send an IPFS hash function to the core service component 402 for further sending the IPFS hash function to EHR synchronization service 412. The EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view the EHR data 502. In such a scenario, the user may first send a signed transaction to a RPC component 404 for granting permission to the hospital representative to view the EHR data 502. Once the permission is granted, the signed transaction may be added to the quorum blockchain node 414 and a new smart contract will be created for a blockchain corresponding to the hospital representative. After adding the signed transaction, the hospital representative may be able to view the EHR data 502 of the user, on a device.

In various embodiments, to view the EHR data 502 on the device, the HIE system 102 may collect the encrypted EHR data 510 from the user's blockchain and may decrypt the encrypted EHR data 510 using patient's RSA private key 518. The HIE system 102 may decrypt the encrypted secret key 516, in an RSA decryptor 520, using RSA private key of the hospital representative. The encrypted EHR data 510 may be decrypted using the RSA public key 512 of the hospital representative, in an AES decryptor 522. This may be done successively. Further, the HIE system 102 may load the decrypted EHR data 502 to the smart contract previously created for the hospital representative.

After loading the decrypted EHR data 502, the RPC component 404 may obtain the signed transaction from the patient's user device and transmit the signed transaction to the quorum blockchain node component 406 of the second level subsystem. The quorum blockchain node component 406 may confirm ownership of the signed transaction and may execute the smart contract for the hospital representative to view the user's data.

In various embodiments, the patient may decline permission for the hospital representative to have access to the EHR data 502. In such an example scenario, the user through a user device may send a signed transaction revoking permission to the RPC component 404. The RPC component 404 may forward the signed transaction to the quorum blockchain node component 406 of the second level subsystem. The quorum blockchain node component 406 may confirm ownership of the signed transaction and may delete the smart contract previously created to allow the hospital representative to have access to the patient's EHR data 502.

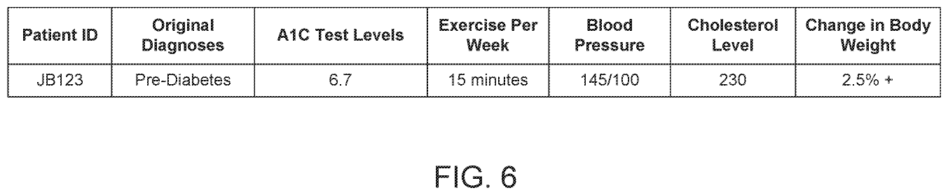

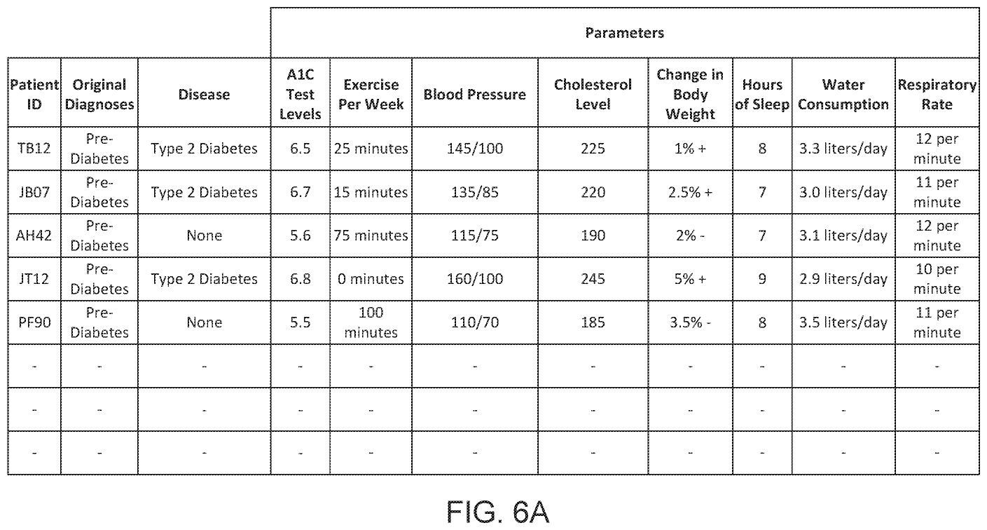

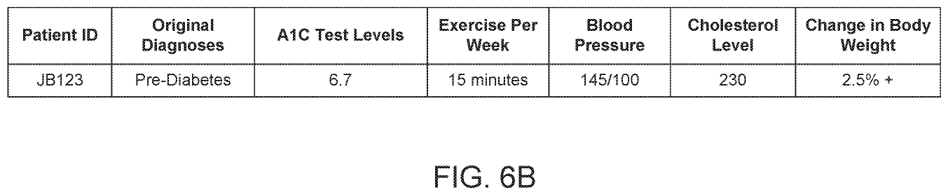

In various embodiments, the treatment database 124 may be configured to store data that passes a predetermined threshold to determine if there is a correlation between the data from the workflow machine learning module 122 by the data collection module 120. As shown in FIG. 6, the treatment database 124 may store patient ID, information related to original diagnoses, and one or more parameters related to the patient. The one or more parameters may include, but not limited to, A1C Test Level, exercise per week, blood pressure, cholesterol level and a change in body weight (i.e., in percentage).

In various embodiments, the patient database 126 may be configured to store information related to patients. The information related to the patients may be viewed on the user device 104 using a public key (received from the physician network device 108). The public key may be combined with a user's private key to access the information. As shown in FIG. 6A, the information stored in the patient database 126 may include, but not limited to, a patient ID, patient original diagnoses, a name of a disease, and one or more parameters related to the patient original diagnosis. In various embodiments, the original diagnosis may reveal pre-diabetes and the disease may be Type 2 diabetes. Further, the one or more parameters may be A1C test, exercise per week (i.e., average minutes), blood pressure, cholesterol level, a change in body weight (percentage since last visit), hours of sleep (i.e., average per night), water consumption, and respiratory rate. It should be noted that the A1C test may indicate an average blood sugar level for a past two to three months. The A1C test may measures a percentage of blood sugar attached to hemoglobin and the oxygen-carrying protein in red blood cells.

In various embodiments, the recommendation database 132 may be configured to store the information related to the patients. The information may be displayed on the physical network GUI for a physician to determine if the correlated data may be used as a recommendation for the patient. Further, as shown in FIG. 6B, the recommendation database 132 may store patient ID, patient original diagnoses, and one or more parameters related to health of the patient. The one or more parameters may include A1C test level, exercise per week, blood pressure, cholesterol level, or change in body weight. In various embodiments, the correlated data may show that the patients with pre-diabetes tend to be diagnosed with diabetes due to one or more parameters, such as, for example, A1C test level, exercise per week, blood pressure, cholesterol levels, and change in body weight. It should be noted that the information may be received from the data collection module 120.

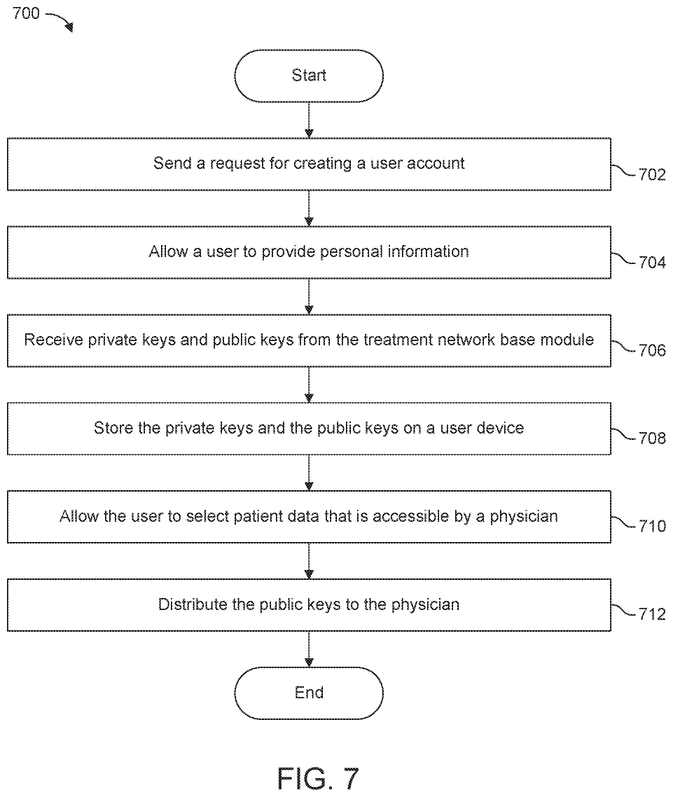

FIG. 7 illustrates an example flowchart showing a method performed by the patient module 128, according to various embodiments. Functioning of the patient module 128 will now be explained with reference to the example flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The patient module 128 may send a request for creating a user account through the treatment network base module 118, at step 702. The patient module 128 may allow the user to provide personal information, at step 704. The personal information may include, for example, an e-mail address, residential address, a telephone number, or an office address. The personal information may be entered via an interface of the user device 104. This may be done successively. The patient module 128 may receive private keys and public keys from the treatment network base module 118, at step 706. This may be done successively. The private keys may be used by the user to access patient data. Further, the public keys may be used by the user to provide an access to the physician to a portion or selected portion of the patient data while blocking the physician from accessing the patient data that is unauthorized by the user. Both the private keys and public keys may be stored on the user device 104.

The patient module 128 may store the private keys and the public keys on the user device 104, at step 708. This may be done successively. The patient module 128 may allow the user to select the patient data that is accessible by the physician, at step 710. The patient module 128 may distribute the public keys to the physician, at step 712. It should be noted that the public keys may be distributed to the physician and may allow access to the portion of the patient data that the user has selected.

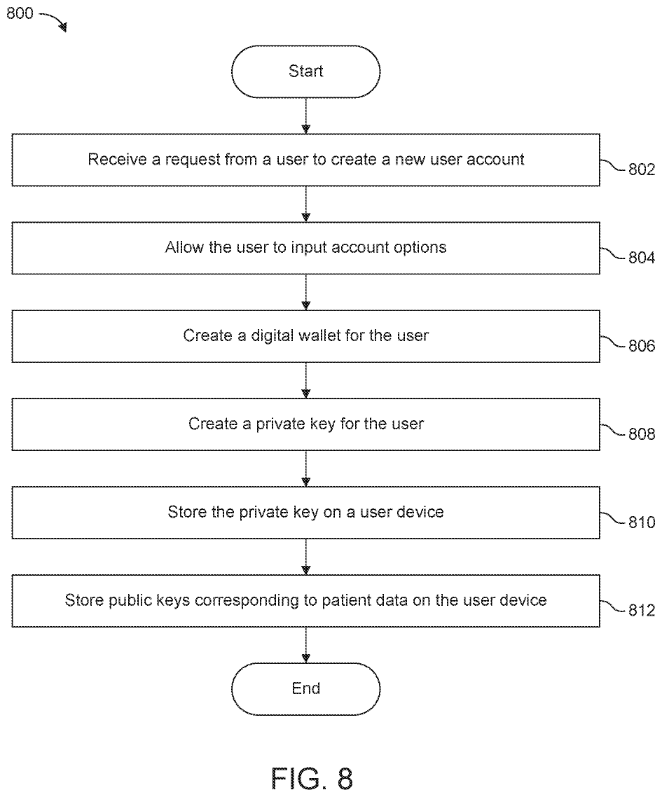

FIG. 8 illustrates an example flowchart 800 showing a method performed by the treatment network base module 118, according to various embodiments. Functioning of the treatment network base module 118 will now be explained with reference to the example flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 8, the treatment network base module 118 may receive a request from a user to create a new user account, at step 802. Upon receiving the request, the treatment network base module 118 may allow the user to input account options, at step 804. The account options may be entered by the user using the interface of the user device 104. In various embodiments, the account option may include personal information, such as an e-mail address, a telephone number, an address, or name of the new user. The treatment network base module 118 may create a digital wallet for the user, at step 806. This may be done successively. In various embodiments, the digital wallet may be, for example, a PTOYNet Ethereum™ blockchain network wallet. The treatment network base module 118 may create a private key for the user, at step 808. This may be done successively. The treatment network base module 118 may store the private key on the user device 104, at step 810. This may be done successively. It should be noted that the private key may be used by the user for modifying or viewing the patient data. The treatment network base module 118 may store public keys corresponding to the patient data on the user device 104, at step 812. In various embodiments, the user may send the public keys to the physician for providing access to the patient data but only those portions of the patient data that are authorized by the user and blocks sections of the patient data.

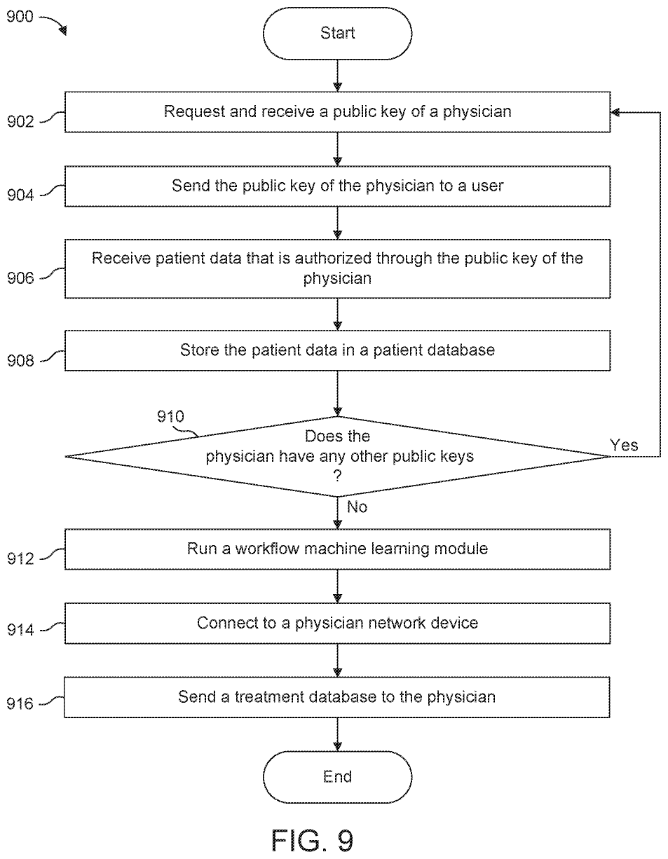

FIG. 9 illustrates an example flowchart 900 showing a method performed by a data collection module 120, according to various embodiments. Functioning of the data collection module 120 will now be explained with reference to the example flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 9, the data collection module 120 may request and receive a public key of the physician, at step 902. In various embodiments, the public key may allow the physician to view a selected portion of the patient data. The data collection module 120 may send the public key of the physician to the user, at step 904. The data collection module 120 may receive the patient data that is authorized through the public key of the physician, at step 906. This may be done successively. It should be noted that the patient data may be accessed by the user using the private key. The data collection module 120 may store the patient data in the patient database 126, at step 908. The data collection module 120 may determine whether the physician has any other public keys, at step 910. This may be done successively. In various embodiments, if the physician is found to have any other public keys, then the data collection module 120 may return the process to step 902. In various embodiments, if the physician does not have any other public keys, then the data collection module 120 may run the workflow machine learning module 122, at step 912.

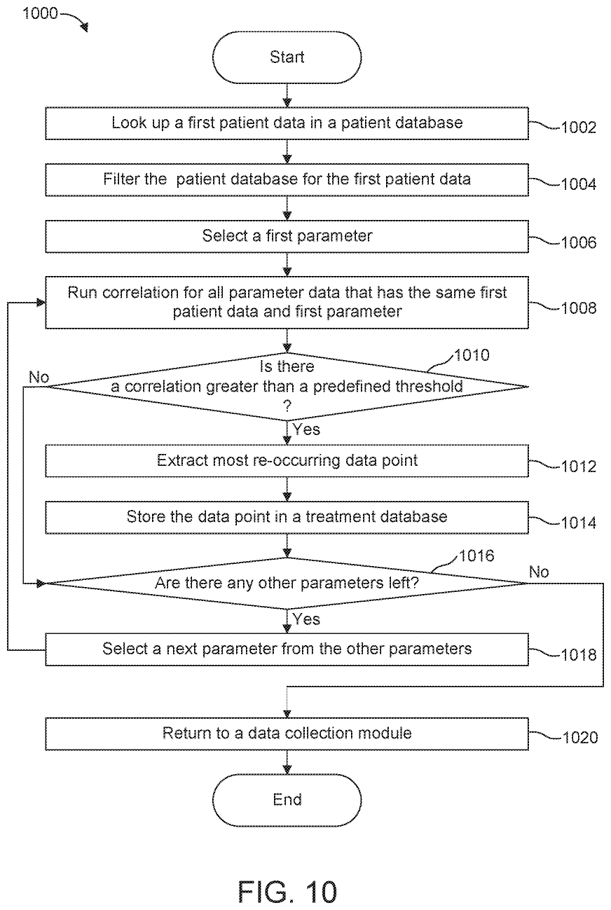

Functioning of the workflow machine learning module 122 will now be explained with reference to the example flowchart 1000 shown in FIG. 10. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The workflow machine learning module 122 may look up a first patient data in the patient database 126, at step 1002. It should be noted that the workflow machine learning module 122 may analyze the authorized patient data received from the user, through the quorum blockchain node component 406. The workflow machine learning module 122 may filter the patient database 126 for the first patient data, at step 1004. This may be done successively. In various embodiments, the first patient data may correspond to a chronic disease. The workflow machine learning module 122 may select a first parameter, at step 1006. This may be done successively. In various embodiments, the first parameter may correspond to A1C test levels. The workflow machine learning module 122 may run correlations for all parameter data that has the same first patient data and first parameter, at step 1008. It should be noted that the correlation may be performed by a correlation engine.

Moreover, the workflow machine learning module 122 may check whether there is a correlation score greater than a predefined threshold, at step 1010. In various embodiments, the predefined threshold may be set as 95%. It should be noted that the correlation may be performed to determine relevancy of the parameter data for the user. In various embodiments, if the correlation is found to be greater than the predefined threshold, the workflow machine learning module 122 may extract most re-occurring data point, at step 1012. In various embodiments, for patients with prediabetes diagnosis, A1C test level at 6.0, and the patients that usually do not exercise (i.e., below 50 minutes per week), the patients are likely to suffer from diabetes. The workflow machine learning module 122 may store the data point in the treatment database 124, at step 1014. This may be done successively. Successive to storage of the data point in the treatment database 124 and while the correlation score does not exceed the predefined threshold, the workflow machine learning module 122 may determine whether any other parameters are left, at step 1016.

In various embodiments, if the other parameters are found, the workflow machine learning module 122 may select a next parameter from the other parameters, at step 1018. The workflow machine learning module 122 may follow the step 1008. This may be done successively. In various embodiments, if no other parameters are found, the workflow machine learning module 122 may return control to the data collection module 120, at step 1020. The data collection module 120 may connect to the physician network device 108, at step 914. This may be done successively. The data collection module 120 may send data stored in the treatment database 124 to the physician, at step 916.

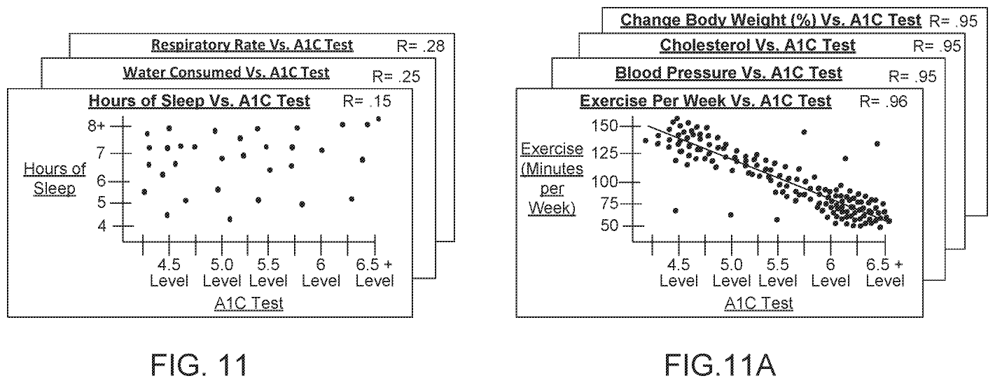

In various embodiments, FIG. 11 illustrates exemplary data stored in the workflow machine learning module 122. Specifically, FIG. 11 illustrates correlations preformed between A1C test and several other parameters. Data that is filtered by the A1C test indicates average blood sugar level of a patient for past two to three months. The workflow machine learning module 122 may measure a percentage of blood sugar attached to hemoglobin and the oxygen-carrying protein in red blood cells. The several other parameters correlated with the A1C test include hours spent sleeping, water consumption, and respiratory rate. In various embodiments, if non-correlated parameters with the A1C test is hours spent sleeping (i.e., on average per night) with, for example, a 15% (i.e., below the 95% threshold), then no correlation may be said to have established and no data points may be stored in the treatment database 124.

In accordance with various embodiments, referring to FIG. 11A, correlations performed between the A1C test and several other parameters are illustrated. The several other parameters used for correlating includes exercise (i.e., average minutes per week), blood pressure, cholesterol level, or change in body weight. In various embodiments, if correlation performed between the A1C test and the exercise results in a correlation score of 96% (i.e., above the 95% threshold), a correlation may be said to be established and a data point may be stored in the treatment database 124. The data point may indicate that patients in the pre-diabetes stage who do not exercise tend to eventually get diagnosed with diabetes. The most re-occurring data point may be extracted and stored in the treatment database 124. In various embodiments, the most re-occurring data point may be related to a patient exercising, for example, below 50 minutes a week, having A1C level of 6.5+, and diagnosed with diabetes.

In various embodiments, the correlated data may be viewed by a healthcare practitioner for determining that the patients with a similar age and same disease usually get diagnosed with diabetes if they are in the prediabetes stage and do not exercise, have abnormal blood pressure, abnormal cholesterol levels, and have increased body weight (i.e., by percentage since last visit). Thereafter, the healthcare practitioner may determine from the correlated data points that the patient may be diagnosed with diabetes.

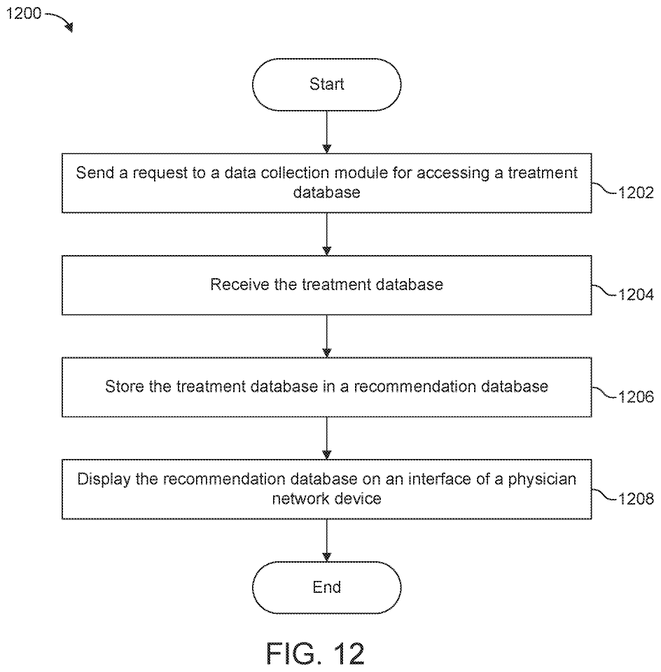

FIG. 12 illustrates an example flowchart 1200 showing a method performed by a physician network base module 130, according to various embodiments. Functioning of the physician network base module 130 will now be explained with reference to the example flowchart 1200 shown in FIG. 12. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 12, the physician network base module 130 may send a request to the data collection module 120 for accessing the treatment database 124, at step 1202. The physician network base module 130 may receive the treatment database 124, at step 1204. This may be done successively. The physician network base module 130 may store the treatment database 124 in the recommendation database 132, at step 1206. This may be done successively. In various embodiments, storing the treatment database 124 in the recommendation database 132 may indicate storing data present in the treatment database 124 into the recommendation database 132. The physician network base module 130 may display the data of the recommendation database 132 on an interface of the physician network device 108, at step 1208. It should be noted that the recommendation database 132 may be displayed on the physician network device 108 to determine whether an adjustment is required to a workflow to improve the original plan of treatment.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for improving treatment of a chronic disease of a patient, comprising:
a. receiving in a health care network, from a physician using a physician network device, a request for accessing at least a portion of a patient data and parameters including a first parameter and a second parameter related to the patient, wherein the patient data is stored over a blockchain;
b. allowing the patient to manage access to the patient data in the blockchain via a user device communicatively coupled with the health care network;
c. correlating, using a correlation engine, the first parameter with patients' data to identify at least two correlated data points relevant for the patient; and
d. sending, to the physician, the correlated data points for being used towards treatment of the chronic disease of the patient.

2. The computer-implemented method of claim 1, further comprising storing the correlated data points relevant for the patient in a treatment database.

3. The computer-implemented method of claim 1, wherein the physician is an individual belonging to one of a hospital, an insurance company, a contract research organization, or a pharmaceutical company.

4. The computer-implemented method of claim 1, further comprising verifying an encrypted key from at least one of the patients or the physician to access the blockchain.

5. The computer-implemented method of claim 1, further comprising providing an encrypted key to at least one of the patients or the physician, to access the blockchain.

6. The computer-implemented method of claim 1, wherein allowing the patient to manage access to the patient data in the blockchain via a user device comprises allowing the patient to select patient data accessible to a physician.

7. The computer-implemented method of claim 1, further comprising sending, to the physician, a datum stored in a treatment database.

8. The computer-implemented method of claim 1, further comprising verifying that the physician has at least one public key to access the blockchain.

9. The computer-implemented method of claim 1, further comprising extracting a most re-occurring data point when a correlation level is greater than a predefined threshold.

10. The computer-implemented method of claim 1, further comprising correlating a second parameter with patients' data when a correlation level is less than a predefined threshold.

11. A system for improving treatment of a chronic disease of a patient comprising:
a. one or more processors; and
b. a memory communicatively coupled to the one or more processors and storing instructions which, when executed by the one or more processors, cause the system to:
receive in a health care network, from a physician using a physician network device, a request for accessing at least a portion of a patient data and parameters related to the patient, wherein the patient data is stored over a blockchain;
allow the patient to manage access to the patient data in the blockchain via a user device communicatively coupled with the health care network;
correlate, using a correlation engine, the parameters with patients' data to identify at least two correlated data points relevant for the patient;
send, to the physician, the correlated data points for being used towards treatment of the chronic disease of the patient; and
store the correlated data points relevant for the patient n a treatment database.

12. The system of claim 11, wherein the physician is an individual belonging to one of a hospital, an insurance company, a contract research organization, or a pharmaceutical company.

13. The system of claim 11, wherein the one or more processors further execute instructions to verify an encrypted key from at least one of the patient or the physician to access the blockchain.

14. The system of claim 11, wherein the one or more processors further execute instructions to provide an encrypted key to at least one of the patient or the physician, to access the blockchain.

15. The system of claim 11, wherein to allow the patient to manage access to the patient data in the blockchain via a user device the one or more processors execute instructions to allow the patient to select patient data accessible to a physician.

16. The system of claim 11, wherein the one or more processors further execute instructions to send, to the physician, a datum stored in a treatment database.

17. A non-transitory, computer readable method storing instructions which, when executed by a processor, cause a computer to perform a method, the method comprising:
a. receiving in a health care network, from a physician using a physician network device, a request for accessing at least a portion of a patient data and parameters related to the patient, wherein the patient data is stored over a blockchain;
b. allowing the patient to manage access to the patient data in the blockchain via a user device communicatively coupled with the health care network;
c. correlating and matching, using a correlation engine, the parameters with patients' data to identify and recommend at least two correlated data points relevant for the patient, said recommendation being a ranked recommendation based on configurable weights associated with said at least two correlated data points;
d. sending recommendation, to the physician, the correlated data points for being used towards treatment of a chronic disease of the patient;
e. storing the correlated data points relevant for the patient in a treatment database; and
f. verifying an encrypted key from at least one of the patient or the physician to access the blockchain.

18. The non-transitory, computer readable medium of claim 17, wherein the one or more processors further execute instructions to provide an encrypted key to at least one of the patients or the physician, to access the blockchain.

19. The non-transitory, computer readable medium of claim 17, wherein to allow the patient to manage access to the patient data in the blockchain via a user device the one or more processors execute instructions to allow the patient to select patient data accessible to a physician.

20. The non-transitory, computer readable medium of claim 17, wherein the one or more processors further execute instructions to send, to the physician, a datum stored in a treatment database.
